(19) United States
(12) Patent Application Publication
(10) Pub. No.: US 2006/0200162 A1
(43) Pub. Date: Sep. 7, 2006

(54) Total knee arthroplasty instruments

(75) Inventors: Toby N. Farling, Warsaw, IN (US); Adam M. Griner, Columbia City, IN (US); W. Matthew Kuester, St. Louis Park, MN (US); Brian E. VanSkyock, Fort Wayne, IN (US)

Correspondence Address: ZIMMER TECHNOLOGY - BAKER & DANIELS, 111 EAST WAYNE STREET, SUITE 800, FORT WAYNE, IN, 46802, US

(73) Assignee: Zimmer Technology, Inc.

(21) Appl. No.: 11/357,825

(22) Filed: Feb. 17, 2006

Related U.S. Application Data
(60) Provisional application No. 60/654,629, filed on Feb. 21, 2005.

(51) Int. Cl. A61B 17/58

(52) U.S. Cl. 606/88

(57) ABSTRACT

A device for implanting a femoral component including a base assembly, a handle slidably attached to base assembly, a locking member slidably coupled to handle, and a locking actuator. Locking member has a locking end extending from handle, through an opening in base assembly and through notch in implant. Locking end has locking lip extending transversely from locking member. Locking member is movable between a locked position, wherein lip is positioned at a first distance from upper surface of base assembly, and an unlocked position, wherein lip is positioned at a second distance from upper surface. When locking member is in the locked position, lip is engagable with proximal surface of implant at a location proximal the notch thereby gripping the implant between the upper surface and lip. Locking actuator is operatively engaged with locking member and is operable to move locking member between locked position and unlocked position.

### PRIORITY REFERENCE

This application claims the benefit of priority under 35 U.S.C. §119(e) to provisional application Ser. No. 60/654,629, entitled TOTAL KNEE ARTHROPLASTY INSTRUMENTS and filed in the names of Toby N. Farling et al. on Feb. 21, 2005.

### BACKGROUND

The present invention relates generally to instruments and methods for performing knee arthroplasty and, more particularly, to instruments for implanting and removing a femoral component or provisional, guiding the reshaping and preparation of the femur, and positioning instruments against the bone.

Orthopedic procedures for the replacement of all, or a portion of, a patient's joint typically require resecting (cutting) and reshaping of the bones of the joint to receive the prosthetic components. For instance, a typical total knee prosthesis has three main components: a femoral component for replacing the distal end of the femur, a tibial component for replacing the proximal end of the tibia, and a bearing insert for replacing the articulating tissue between the femur and the tibia. Procedures for implanting a total knee prosthesis typically involve preparing and reshaping both the distal end of the femur and the proximal end of the tibia prior to implanting the prosthesis components. The amount of bone removed is determined, in part, by the size and type of components being implanted. For instance, patients having a healthy, intact posterior cruciate ligament are often fitted with a “standard” femoral component.

A typical “standard” femoral component includes a bone engaging surface and an opposing articulating surface. The articulating surface forms a pair of posterior condyles, which are spaced apart by an intracondylar notch extending through the femoral component from the bone engaging surface to the articulating surface. The bone engaging surface is shaped to wrap around the prepared end of the femur and includes a posterior portion, an anterior portion, a distal portion, an anterior chamfer portion, a posterior chamfer portion, and trochlear portion. Accordingly, reshaping the distal end of the femur to receive a “standard” femoral component often involves making several cuts of the distal end of the femur including a distal (resecting) cut, an anterior cut, a posterior cut, a trochlear cut, an anterior chamfer cut, and a posterior chamfer cut to provide the distal end of the femur with a shape complementary to the bone engaging surface of the “standard” femoral component.

On the other hand, patients having damaged posterior cruciate ligaments may be fitted with a “posterior stabilized” femoral component. A “posterior stabilized” femoral component includes a bone engaging surface and articulating surface similar to that of the “standard” femoral component. The “posterior stabilized” femoral component differs, in part, from the “standard” femoral component in that it includes a femoral box protruding outwardly from the distal portion of the bone engaging surface and extending along the edge of the intracondylar notch. Accordingly, when preparing the femur to receive a “posterior stabilized” femoral component, a femoral box cut must be made in the distal end of the femur to accommodate the femoral box.

In a recently developed femoral implant, disclosed in U.S. Pat. No. 6,123,729 to Insall et al., entitled Four Compartment Knee, the articulating surface has been extended to increase the width of the posterior condyles measured from articulating surface to bone engaging surface and, thereby, provide superior condyles. This design provides a greater range of flexion and may be referred to as a “flex” femoral component. In this case, the distal portion of the bone engaging surface is decreased and additional bone may need to be removed to receive the “flex” component.

Cut guides have been developed to guide a cutting instrument in making the necessary cuts in the distal end of the femur and the proximal end of the tibia. Conventional cut guides are often in the form of blocks having permanently positioned slots therein for receiving and guiding the cutting instrument. Different sized and shaped cut guide blocks are provided to correspond to different sizes and styles of prostheses and to achieve the different cuts. In addition, oftentimes multiple cut guide blocks are required to make all the necessary cuts. Accordingly, shaping of the distal end of the femur and the proximal end of the tibia may require consecutive placement and removal of multiple cut guide blocks on the bone. Furthermore, proper resection and shaping of both the femur and the tibia requires proper alignment of the cut guides. In cases where the surface of the bone is irregular, it may be difficult to accurately position the cut block on the surface of the bone.

Additionally, minimally invasive surgical techniques are becoming increasingly popular. Minimally invasive surgical techniques employ, among other things, considerably smaller incisions and tighter working spaces than historical techniques in an effort to reduce trauma to nearby tissue and, thereby, accelerate post-operative recovery. Proper alignment and implantation of the implant components and provisionals requires reliable grasping and manipulation of the implant components and provisionals in a tight, small space.

Accordingly, a need remains for minimally invasive surgical instruments that allow the manipulation and placement of prosthesis components and provisionals on the bone through a small incision and in a small surgical site. Furthermore, a need remains for improved cut guides that minimize the installation and removal of multiple cut guides on the femur, and improve the efficiency of the reshaping procedures. Finally, a need remains for instruments that aid in the proper alignment and positioning of instruments, such as cut guides, against the surface of the bone.

### SUMMARY

The present invention provides instruments and methods for performing knee arthroplasty including instruments for implanting and removing a femoral component or provisional, guiding the reshaping and preparation of the femur, and positioning instruments against the bone.

In one aspect, the present invention provides a device for inserting an implant into an end of a bone and/or for removing the implant from the end of the bone. The implant includes a proximal surface configured to be positioned against the end of the bone and an opposite distal surface. A notch extends through the implant from the proximal surface to the distal surface. The device generally includes a base assembly, a handle, a locking member and a locking actuator. The base assembly includes an upper bearing surface configured to bear against the distal surface of the implant. An opening extends through the base assembly and is positioned to align with the notch when the upper bearing surface bears against the distal surface of the implant. The handle has a first end slidably coupled to the base assembly and an opposite second end. The locking member is slidably coupled to the handle and has a locking end extending from the handle and through the opening of the base assembly. The locking end extends through the notch of the implant when the upper bearing surface bears against the distal surface of the implant. The locking end has a locking lip extending transversely from the locking member. The locking member is movable between a locked position, wherein the lip is positioned at a first distance from the upper bearing surface, and an unlocked position, wherein the lip is positioned at a second distance from the upper bearing surface. The second distance is greater than the first distance. When the locking member is in the locked position, the lip is engagable with the proximal surface of the implant at a location proximal the notch thereby gripping the implant between the upper bearing surface and the lip. The locking actuator is operatively engaged with the locking member and is operable to move the locking member between the locked position and the unlocked position.

In another aspect, the present invention provides a cut guide assembly for use in shaping the end of a femur to receive a femoral component. The end of the femur has an anterior side, a posterior side and a distal end. The femoral component has a bone engaging surface including a posterior surface, an anterior surface, a distal surface, an anterior chamfer surface extending at a first angle between the anterior surface and the distal surface, a posterior chamfer surface extending at a second angle between the posterior surface and the distal surface, and a trochlear surface extending between the anterior surface and the distal surface and having a first geometry. The femoral component optionally includes a femoral box projecting outwardly from the bone engaging surface and having a second geometry. The distal surface defines a width. The femoral component defines either a first length or a second length extending between the anterior and posterior surfaces.

The cut guide assembly includes a chamfer guide and a trochlear guide. The chamfer guide includes a distal portion and an anterior portion. The distal portion includes opposing medial and lateral ends, opposing anterior and posterior edges, and opposing distal and proximal faces extending between the opposing ends and opposing edges. The distal portion defines a medial-lateral width extending between the medial and lateral ends. The medial-lateral width has a size corresponding to the width of the distal surface of the femoral component. The proximal face is configured to bear against the distal end of the femur. The distal portion has an anterior chamfer slot and a posterior chamfer slot extending therethrough. The anterior and posterior chamfer slots each defines an angle relative to the distal face and corresponding to the first and second angles of the anterior and posterior chamfer surfaces. The distal portion includes a pair of spaced apart arms defining a box-cut guide opening therebetween. The box-cut guide opening has a shape corresponding to the second geometry. The anterior portion of the chamfer guide extends from the proximal face of the distal portion adjacent anterior edge. The anterior portion has an inside surface configured to bear against the anterior side of the femur. The chamfer guide defines a first anterior-posterior length extending between the inside surface of anterior portion and the posterior edge of distal portion. The first anterior-posterior length has a size corresponding to the first length of the femoral component.

The trochlear guide has a lower surface and an upper surface. The lower surface has a projection extending outwardly therefrom. The projection is removably nested in the box-cut guide opening to interconnect the trochlear guide to the chamfer guide. The trochlear guide includes a trochlear cut guide opening extending through the trochlear guide from the upper surface to the lower surface of the projection. The trochlear guide has a second posterior edge overlying the posterior edge of the distal portion when the trochlear guide is interconnected with the chamfer guide. The guide assembly defines a second anterior-posterior length extending between the inside surface of the anterior portion of the chamfer guide and the second posterior edge of the trochlear guide. The second anterior-posterior length has a size corresponding to the second length of the femoral component.

In one embodiment of the cut guide assembly, the trochlear cut guide opening is in the form of a captured slot. In addition, the distal portion may include at least one threaded handle receiving opening. The trochlear guide may include at least one handle receiving hole extending therethrough and aligned with the at least one threaded handle receiving opening of the distal portion. The anterior portion may include at least one fastener opening extending therethrough. The distal portion may include at least one drill guide bore extending therethrough.

In yet another aspect, the present invention provides a device for positioning an implant or instrument against a bone. The implant or instrument includes a threaded receiving opening extending therethrough. The positioning device includes a sleeve extending between a first end and an opposite second end. The sleeve has a passage extending between the first end and the second end. The first end defines a recess coaxial with and in communication with the passage. The second end defines an opening coaxial with the passage. The sleeve has a threaded external surface threadedly engagable with the threaded device receiving opening.

In a particular embodiment, the recess of the positioning device has a hexagonal cross sectional shape. The positioning device may also include a pin sized to extend through the passage and into the bone to secure the implant against the bone.

In another aspect, the present invention provides a device for positioning an implant or instrument against a bone, including an elongate sleeve extending between a first end and an opposite second end and defining an axis. The sleeve has a passage extending therethrough from the first end to the second end along the axis. The first end has a tool-engaging cross-sectional shape. The sleeve has a threaded external surface configured to threadedly engage with and extend through the threaded opening. A drive tool engages the first end and drives the sleeve into and out of engagement with the threaded opening.

In a particular embodiment, one of the first end and the drive tool includes a female engagement member, and the other of the first end and the drive tool comprises a male engagement member. The male engagement member is removably received within the female engagement member to lock the drive tool to the sleeve. The female engagement member may be in the form of a recess having a hexagonal cross-sectional shape, and the male engagement member may be in the form of a projection having a hexagonal cross-sectional shape.

### BRIEF DESCRIPTION OF THE DRAWINGS

The above mentioned and other features and objects of this invention, and the manner of attaining them, will become more apparent and the invention itself will be better understood by reference to the following description of an embodiment of the invention taken in conjunction with the accompanying drawings, wherein:

FIG. 1 is a perspective view of an inserter device according to one embodiment of the present invention wherein the locking lever is in a secured position;

FIG. 2 is a perspective view of the inserter device of FIG. 1 wherein the locking lever is in a released position;

FIG. 3 is a front or anterior view of the inserter device of FIG. 1;

FIG. 4 is a back or posterior view of the inserter device of FIG. 1;

FIG. 5 is a back perspective view of part of the inserter device of FIG. 1;

FIG. 6 is a front perspective view of part of the inserter device of FIG. 1;

FIG. 7 is an exploded view of the inserter device of FIG. 6;

FIG. 7A is a perspective view of the base of the inserter device of FIG. 1;

FIG. 7B is a perspective view of the bearing pad of the inserter device of FIG. 1;

FIG. 8 is a side view of part of the inserter device of FIG. 1;

FIG. 8A is a side view of part of the inserter device of FIG. 1 wherein in the locking lever is between the released position and the secured position;

FIG. 9 is a side view of part of the inserter device of FIG. 2;

FIG. 10 is a bottom perspective view of the inserter device of FIG. 1;

FIG. 11A is a back view of the inserter device of FIG. 1;

FIG. 11B is a sectional view of the device of FIG. 11A taken along lines 11B-11B;

FIG. 12 is a side view of the inserter device of FIG. 1 locked to a femoral implant;

FIG. 13 is a top perspective view of the inserter device of FIG. 12;

FIG. 14A is a top perspective view of a femoral cut guide assembly in accordance with one embodiment of the present invention;

FIB. 14B is a bottom perspective view of the femoral cut guide assembly of FIG. 14A;

FIG. 15 is a bottom perspective view of the chamfer guide of the guide assembly of FIG. 14A;

FIG. 16 is a top perspective view of the chamfer guide of FIG. 15;

FIG. 17 is a bottom perspective view of the trochlear guide of the guide assembly of FIG. 14;

FIG. 18 is a top perspective view of the trochlear guide of FIG. 17;

FIG. 19A is a perspective view of a femoral component of a standard (cruciate retaining) knee implant;

FIG. 19B is a perspective view of a femoral component of a posterior stabilized knee implant;

FIG. 19C is a perspective view of a femoral component of yet another knee implant;

FIG. 20 is a perspective view of the chamfer guide of FIG. 15 mounted on the distal end of a femur;

FIG. 21 is a perspective view of the guide assembly of FIG. 14A mounted on the distal end of the femur;

FIG. 22 is a perspective view of a positioning device in accordance with one embodiment of the present invention;

FIG. 23 is an end view of the positioning device of FIG. 22; and

FIG. 24 is a perspective view of the positioning device of FIG. 22 coupled with a tibial cut guide.

Corresponding reference characters indicate corresponding parts throughout the several views. Although the drawings represent embodiments of the present invention, the drawings are not necessarily to scale and certain features may be exaggerated in order to better illustrate and explain the present invention. Although the exemplification set out herein illustrates embodiments of the invention, in several forms, the embodiments disclosed below are not intended to be exhaustive or to be construed as limiting the scope of the invention to the precise forms disclosed.

### DETAILED DESCRIPTION

The embodiments hereinafter disclosed are not intended to be exhaustive or limit the invention to the precise forms disclosed in the following description. Rather the embodiments are chosen and described so that others skilled in the art may utilize its teachings.

The present invention will now be described with reference to the attached figures. The description below may include references to the following terms: anterior (at or near the front of the body, as opposed to the back of the body); posterior (at or near the back of the body, as opposed to the front of the body); lateral (at or near the left side of the body, farther from the midsagittal plane, as opposed to medial); medial (at or near the middle of the body, at or near the midsagittal plane, as opposed to lateral); proximal (toward the beginning, at or near the head of the body, as opposed to distal) and distal (further from the beginning, at or near the foot of the body, as opposed to proximal).

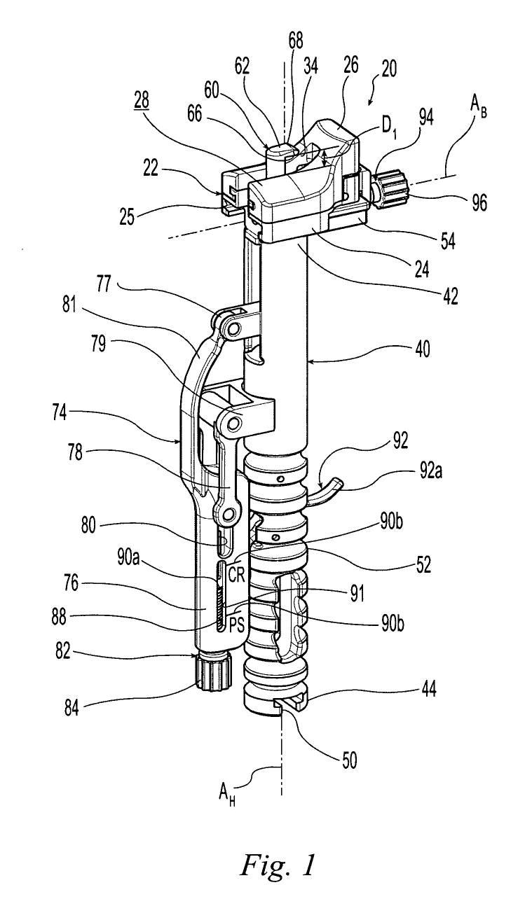

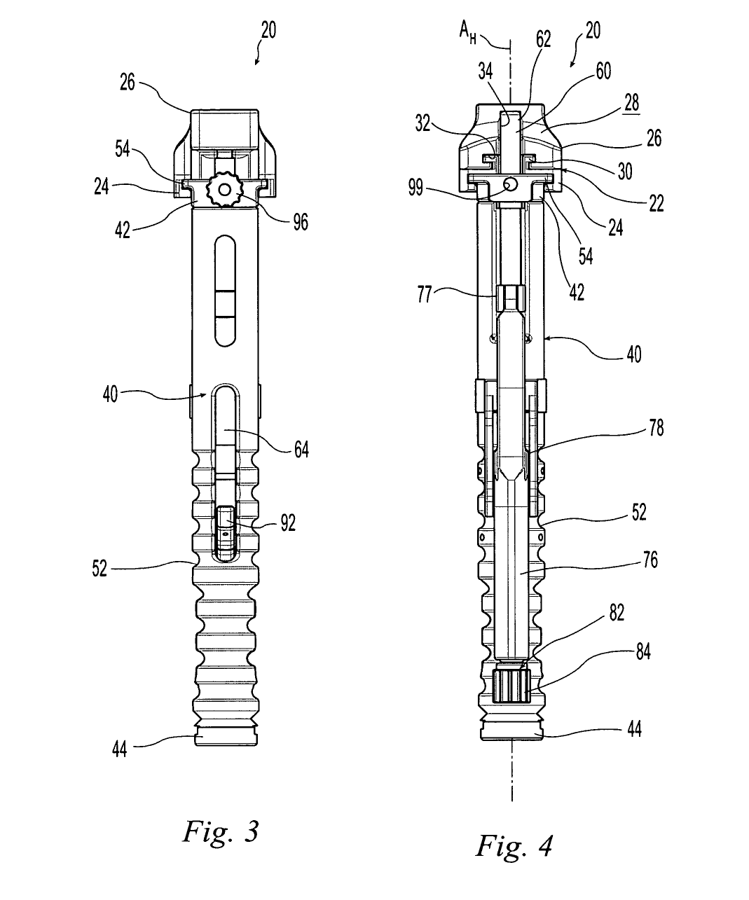

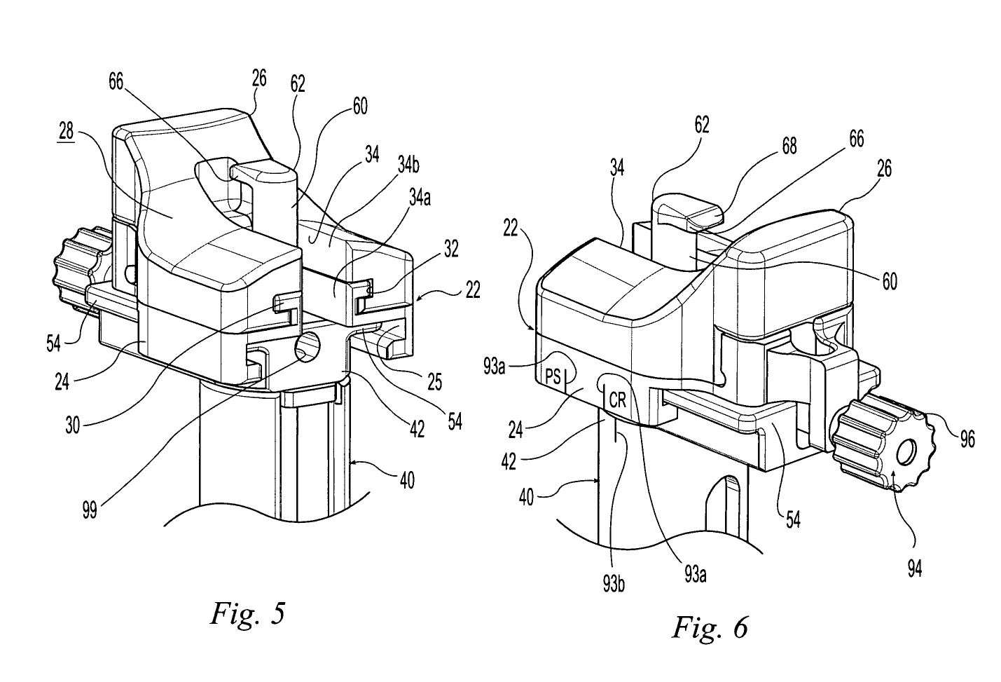

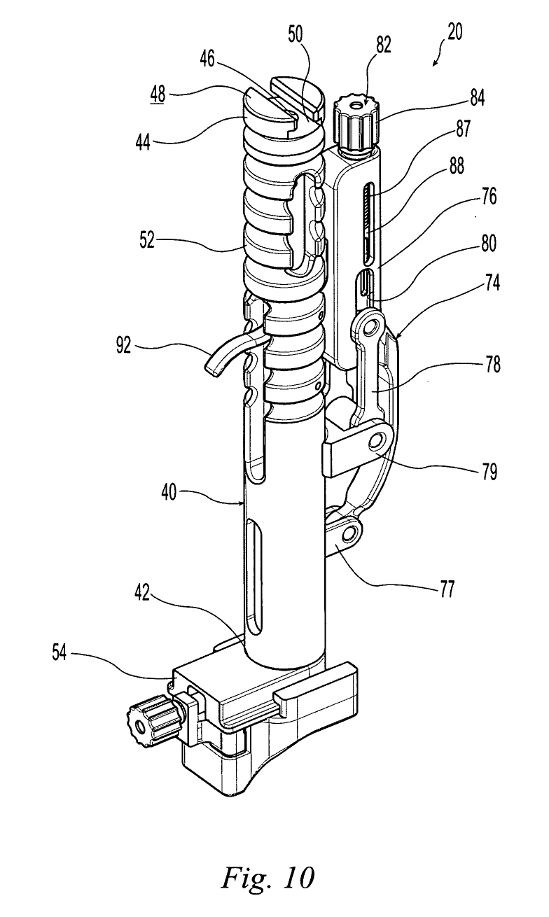

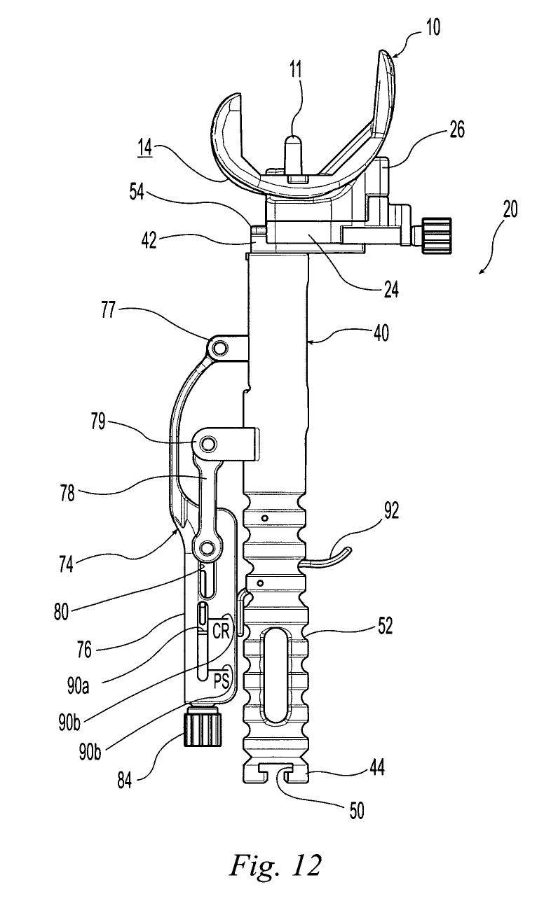

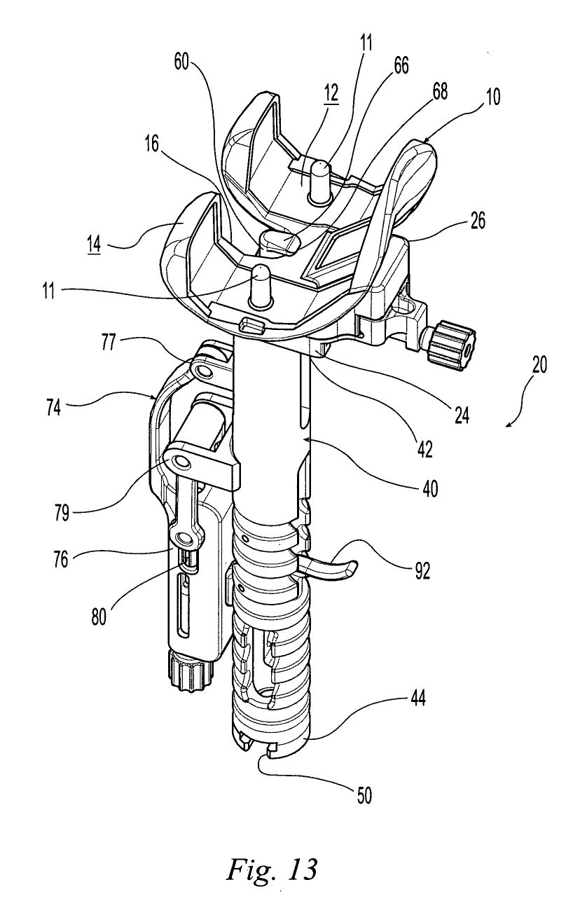

Referring first to FIGS. 1-13, inserter device 20, according to one embodiment of the present invention, is illustrated. As shown in FIGS. 12 and 13 and described in further detail below, device 20 is adapted to facilitate the insertion of femoral component 10 of a knee implant into the distal end of the femur and/or the removal of the femoral component from the distal end femur. It should be understood that inserter device 20 may also be used to mount a provisional femoral component on the femur and/or remove the same from the femur. In addition, the present invention contemplates devices similar to device 20 that may be adapted to mount other implant components to and/or remove such components from a bone. For instance, devices of the present invention may be adapted to mount a tibial component, shoulder implant component or hip implant component to a bone.

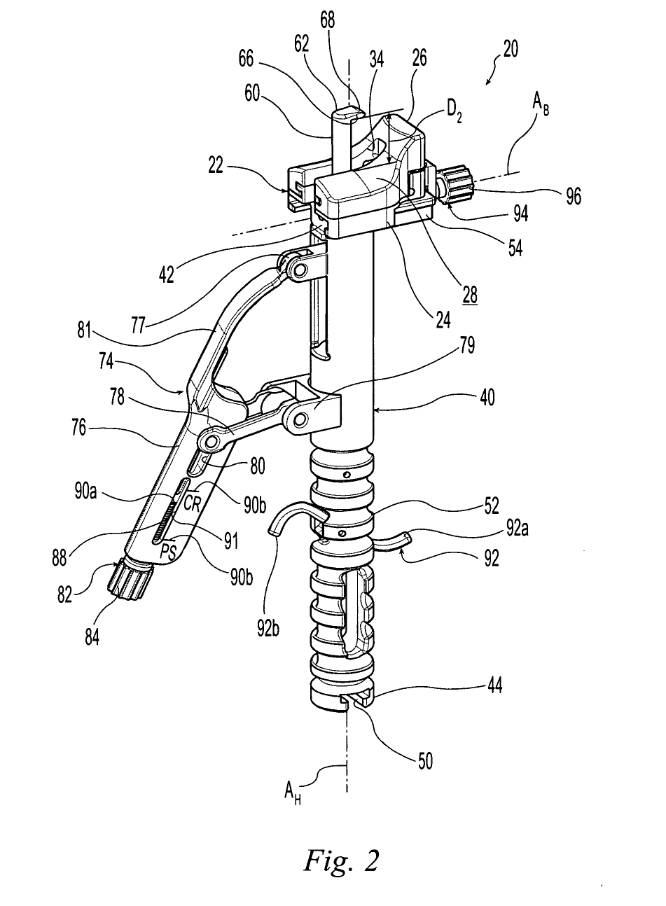

Referring specifically to FIGS. 1 and 2, device 20 generally includes base assembly 22, elongate handle 40 slidably coupled at one end to base assembly 22, locking member 60 slidably coupled to handle 40 and locking actuator assembly 74 operably coupled to locking member 60 and operable to slide locking member 60 relative to handle 40.

More specifically, as illustrated in FIGS. 1-2 and 5-7B, base assembly 22 includes base 24 and bearing pad 26 removably engaged to base 24. It should be noted that, although base 24 and bearing pad 26 are shown and described as two separate and separable pieces and benefits of such a configuration are discussed below, base 24 and bearing pad 26 may be formed integrally with one another to form a single unit (i.e. base 24 and bearing pad 26 may be one piece). Base 24 may be formed of any rigid surgical grade material including metals and plastics. Base 24 includes elongate engagement member 25 in the form of a T-shaped channel defined in its lower surface. As discussed in further detail below, channel 25 facilitates the sliding engagement between base 24 and handle 40. Base 24 further includes T-shaped projection 30 extending from its upper surface. Projection 30 facilitates engagement with bearing pad 26, as is discussed below. Base 24 also includes hole 97 for receiving base adjustment member 94, as discussed in further detail below.

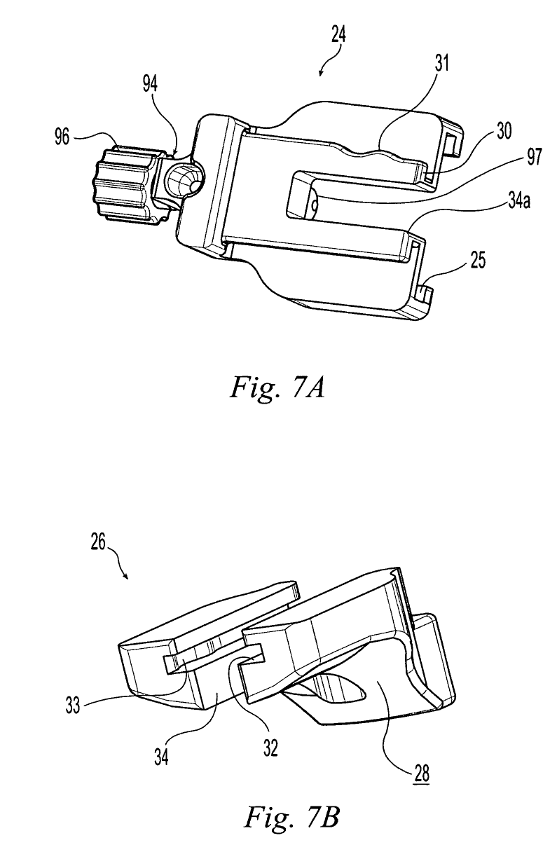

Referring still to FIGS. 1-2 and 5-7B, bearing pad 26 includes upper bearing surface 28, which is contoured to mate with articulating surface 14 of femoral component 10 (FIG. 13). Bearing pad 26 may be formed of any surgical grade material including metals and/or plastics. It is particularly beneficial to construct bearing pad 26 of a material that provides a non-abrasive bearing surface 28, so as to avoid damaging articulating surface 14 of femoral implant 10. Bearing pad 26 further includes T-shaped channel 32 defined in the lower surface opposite bearing surface 28. Channel 32 is configured to mate with and receive projection 30 of base 24 to thereby removably couple bearing pad 26 to base 24. As shown in FIGS. 7A and 7B, projection 30 and channel 32 may include pip 31 and depression 33, respectively. Pip 31 and depression 33 are configured to mate with one another and thereby lock projection 30 in channel 32. In addition, when assembling bearing pad 26 to base 24 the mating of pip 31 with depression 33 provides the user with a positive sensation indicating that bearing pad 26 is properly mounted on base 24.

As shown in FIGS. 7A and 7B, each of base 24 and bearing pad 26 includes opening or notch 34a, 34b, respectively, extending therethrough. As illustrated in FIGS. 1, 2, 4 and 5, when base 24 is coupled with bearing pad 26, notches 34a, 34b are aligned with one another to form opening or notch 34 extending through base assembly 22.

Although bearing pad 26 is attached to base 24 by mating T-shaped channel 32 and projection 30, alternative means of coupling may be used. For instance, bearing pad and base may include mating features having various shapes including a dove-tail shape. Also, the female mating feature (e.g. channel 32) and male mating feature (e.g. projection 30) need not be defined in bearing pad 26 and base 24, respectively. Rather, the female mating feature may reside on base, while the male mating feature may reside in bearing pad. Furthermore, bearing pad 26 need not be secured to base 24 using a male-female engagement, rather bearing pad may be snap-fit, press-fit, glued, welded or otherwise affixed to base 24.

Turning now to FIGS. 1-4 and 10-11B, elongate handle 40 includes first end 42 and opposite second end 44. Handle 40 defines handle axis A_{H} and includes central passage 46 extending therethrough along handle axis A_{H} from first end 42 to second end 44. At second end 44, handle 40 includes impact receiving surface 48, which is adapted to receive and transfer force from a force delivering object such as a hammer or mallet. Second end 44 of handle 40 also includes female engagement feature 50 in the form of a T-shaped slot, which is adapted to couple with a complementary male engagement feature of an extractor instrument (not shown), such as a slap-hammer. Although engagement feature 50 is illustrated as a T-shaped slot, the engagement feature may have any shape or design. Furthermore, if female and male engagement members are utilized to couple the extractor instrument to handle 40, the female and male engagement features may be defined in either the extractor instrument or handle 40. Handle 40 also includes gripping portion 52 near second end 44. Gripping portion 52 is designed to facilitate the user's grip of device 20 and may be in the form of multiple ribs, as shown in FIGS. 1 and 2. Alternatively, gripping portion 52 may be in any form that would enhance the user's grip of device 20.

Referring now to FIGS. 1-2 and 5-6, first end 42 of handle 40 includes male engagement feature 54 in the form of a T-shaped projecting track extending along and defining base axis A_{B}. Base axis A_{B} is transverse to handle axis A_{H}. Male engagement feature 54 of handle 40 is configured to mate with female mating feature (channel) 25 of base 24 to slidably couple base assembly 22 to first end 42 of handle 40. When coupled to handle 40, base assembly 22 is slidable along base axis A_{B}, and notch 34 of base assembly 22 is aligned with and in communication with central passage 46 of handle 40.

Referring to FIGS. 1-7 and 11B, locking member 60 is in the form of an elongate rod and includes locking end 62 and free end 64. Locking member 60 is slideably and partially disposed within central passage 46 of handle 40 such that free end 64 is disposed within central passage 46 and locking end 62 extends outwardly from first end 42 of handle 40. When base assembly 22 is coupled to handle 40, locking end 62 of locking member 60 extends through notch 34 of base assembly 22. Locking member 60 is slidable relative to handle 40 along handle axis A_{H}.

Locking member 60 includes locking hook or lip 66, which extends transversely from locking end 62 and has chamfered edge 68. Locking member 60 is slideable along axis A_{H} between a first locked position shown in FIG. 1 and a second unlocked position shown in FIG. 2. As illustrated in FIG. 1, in the locked position, locking member 60 is positioned such that locking lip 66 is positioned at first distance D₁ from upper bearing surface 28 of bearing pad 26. As shown in FIG. 2, in the unlocked position, locking member 60 is positioned such that locking lip 66 is positioned at second distance D₂ from upper bearing surface 28. As is discussed in further detail below, second distance D₂ is greater than first distance D₁.

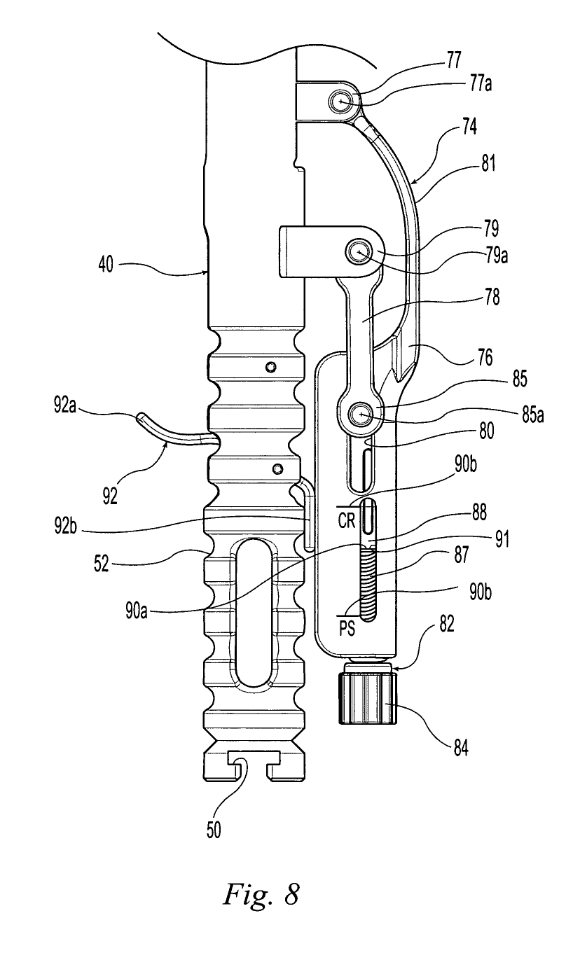

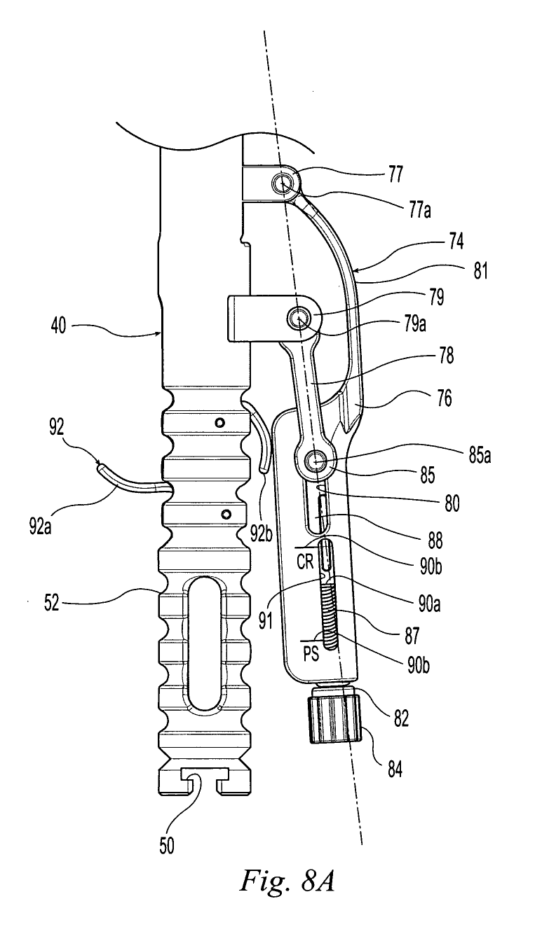

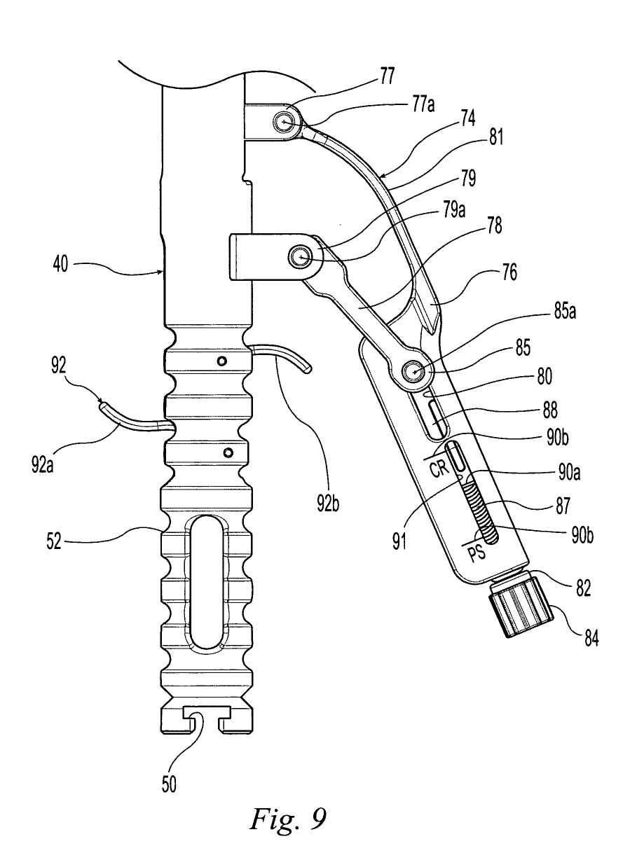

Turning now to FIGS. 1-4 and 8-11B, locking actuator assembly 74 is operably coupled to locking member 60 and is operable to move locking rod 60 between the locked and unlocked positions. Locking actuator assembly 74 includes locking lever 76 pivotally coupled at one end to locking member 60 by hinge joint 77. Locking lever 76 pivots at hinge joint 77 about pivot point 77a. Locking lever 76 is also pivotally coupled to handle 40 via pivot arm or bracket 78 and hinge joint 79. More particularly, locking lever 76 includes elongate slot 80 extending therethrough. Pivot bracket 78 is slideably and pivotally engaged at one end to hinge 85 in slot 80 of lever 76 and pivots relative to locking lever 76 about pivot point 85a. At the opposite end, pivot bracket 78 is pivotally engaged to handle 40 at hinge 79 and pivots about pivot point 79a. Locking lever 76 also includes deflecting portion 81 extending from hinge joint 77 to slot 80. As is discussed in further detail below, locking lever 76 is pivotable between a secured position, shown in FIGS. 1 and 8, and a released position shown in FIGS. 2 and 9. In the secured position, locking lever 76 locks locking member 60 in the locked position. In the released position, locking lever 76 releases locking member 60 from the locked position.

Turning to FIGS. 1-2 and 8-9, locking actuator assembly 74 also includes height adjustment member 82. Height adjustment member 82 is operably coupled to locking rod 60 and is operable to adjust first and second distances D₁ and D₂. More specifically, height adjustment member 82 is in the form of a bolt having knob 84 at one end and threaded portion 87 at the opposite end (FIGS. 8 and 9). Threaded portion 87 is captured within locking lever 76 and is threadedly received in sleeve 88. Sleeve 88 is disposed within locking lever 76 and is engaged at one end to pivot bracket 78 in the area of slot 80. Guide markings 90a, 90b are provided on sleeve 88 and locking lever 76, respectively. Guide marking 90a is viewable through slot 91 in locking lever 91. Guide markings 90b correspond to the implants that may be installed or removed using insertion device 20. For instance, one of markings 90b corresponds to the first and second distance D₁, D₂ settings needed for a standard, cruciate retaining (CR) femoral component, while the other of markings corresponds to the first and second distance D₁, D₂ settings needed for a posterior stabilized (PS) femoral component. Alternatively, markings 90a, 90b may provide any useful guiding information including, for example, implant size or measurements.

Referring still to FIGS. 1-2 and 8-9, spring release lever 92 is pivotally coupled to handle 40 and includes trigger 92a and release tab 92b. Release tab 92b is positioned adjacent to and abuts locking lever 76. Release lever 92 is spring biased to the position shown in FIGS. 1 and 8, but is pivotable to the position shown in FIGS. 2 and 9. As is discussed in further detail below, release lever 92 is adapted to force locking lever 76 to the released position when pivoted to the position shown in FIGS. 2 and 9.

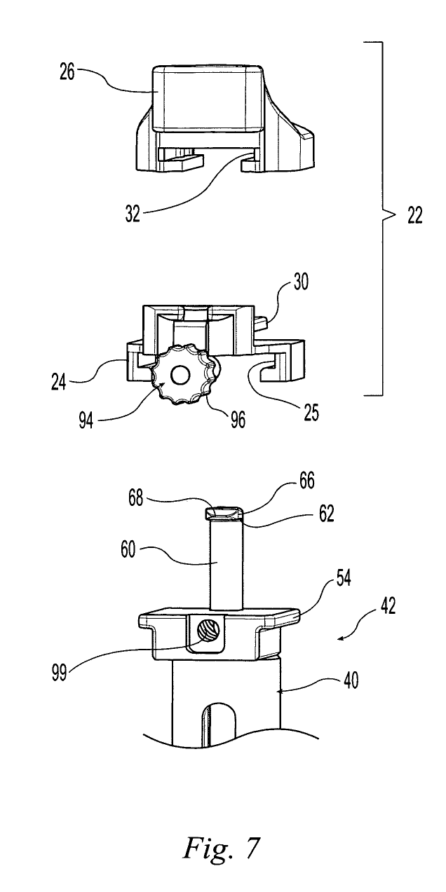

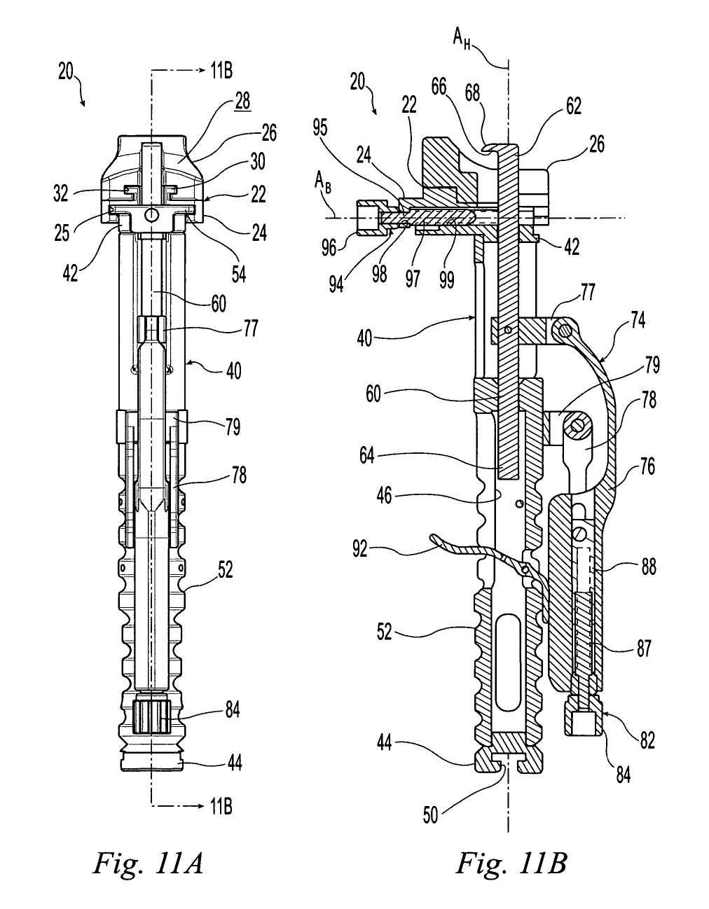

Referring now to FIGS. 1-2, 5-7B and 11B, inserter device 20 also includes base adjustment member 94. Base adjustment member 94 includes knob 96 at one end, threaded portion 97 at the opposite end, and neck portion 95 between knob 96 and threaded portion 97. Neck portion 95 is rotatably captured in hole 98 in base 24 (FIG. 11B). Hole 98 is in alignment with base axis A_{B}. Threaded portion 97 is received in threaded passage 99 in handle 40 (FIGS. 7 and 11B). Threaded passage 99 extends into male engagement feature 54 of handle 40 along base axis A_{B}. Guide markings 93a, 93b are provided on base 24 and handle 40, respectively (FIG. 6) to guide the positioning of base assembly 22 along base axis A_{B}.

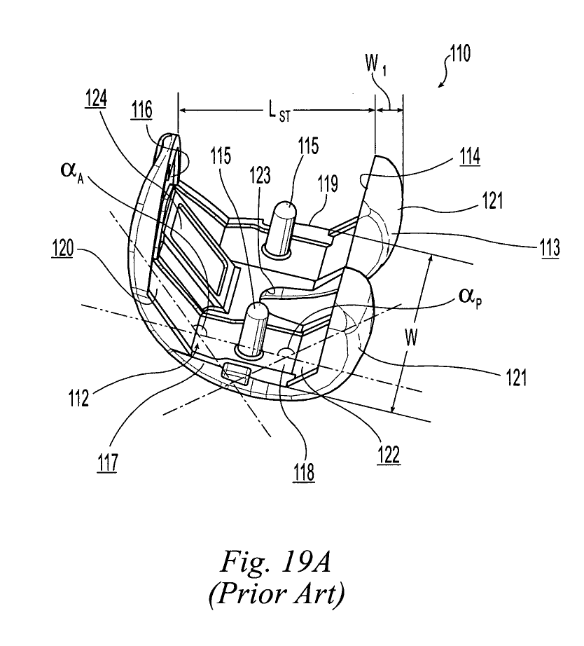

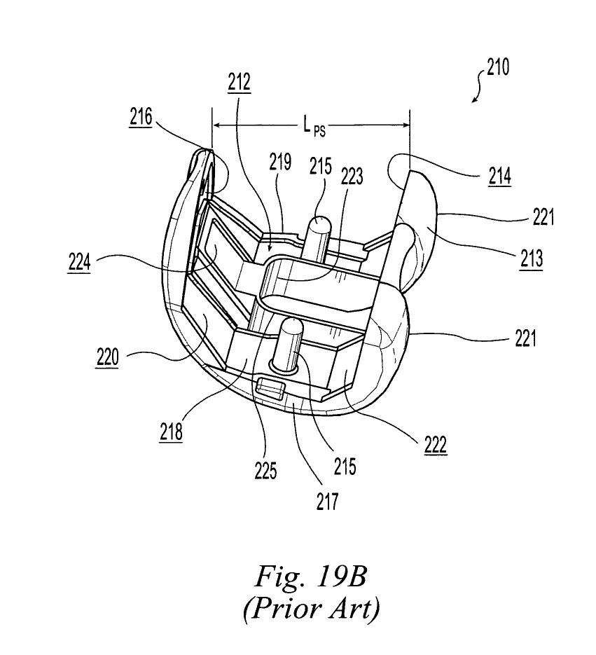

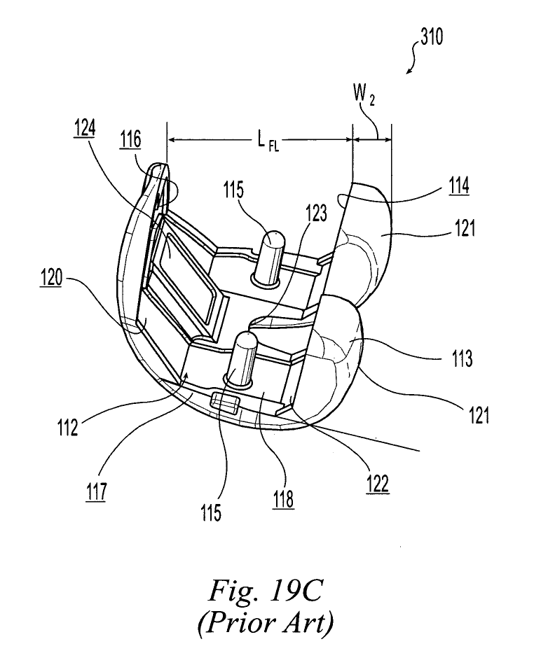

Referring now to FIGS. 1-2, 7, 7A-B, 8-9 and 12-13, the operation of inserter device 20 will now be described. As noted above, inserter device 20 may be used to mount a femoral component on the prepared distal end of a femur. For exemplary purposes, the use of inserter device 20 to implant a cruciate retaining (“CR”) femoral component, such as femoral component 10, is illustrated and described below. However, it should be noted that inserter device 20 may also be used to implant various styles and sizes of femoral components including a posterior stabilized (“PS”) femoral component, such as that shown in FIG. 19B. Returning to FIGS. 12 and 13, femoral component 10 includes bone engaging surface 12 and opposing articulating surface 14. Notch 16 extends through femoral component 10 from bone engaging surface 12 to articulating surface 14. Femoral component 10 also includes a pair of posts 11 extending outwardly from bone engaging surface 12.

First, the distal end of the femur (not shown) is prepared to receive femoral component 10 using any known means including, for instance, the methods described and illustrated in U.S. Publication No. 2004/0153066 to Coon et al, filed as U.S. patent application Ser. No. 10/356,404, entitled Instruments for Knee Surgery and Method of Use, assigned to the assignee of the present application and hereby incorporated by reference. Preparation of the femur typically involves making distal, anterior, posterior and/or chamfer cuts to give the distal end of the femur a shape complementary to bone engaging surface 12. In addition, holes may be drilled into the distal end of the femur (not shown) to receive posts 11 of femoral component 20.

Once the distal end of the femur is prepared, inserter device 20 is used to insert femoral component 10 into and mount femoral component 10 onto the distal end of the femur. First, bearing pad 26 is selected and mounted to base 24 to form base assembly 22. In the illustrated embodiment bearing pad 26 is a separate and distinct part from base 24. Accordingly, varying shapes and sizes of bearing pads may be available and the appropriate bearing pad 26 may be selected from these available pads based on the size and type of component being implanted. In addition, bearing pad 26 is replaceable in the event bearing pad 26 becomes damaged or a different bearing pad is needed.

Turning to FIGS. 1-2 and 5-7B, bearing pad 26 is mounted to base 24 by sliding T-shaped projection 30 of base 24 into T-shaped channel 32 in bearing pad 26. Projection 30 is slid into channel 32 until pip 31 (FIG. 7A) of projection 30 mates with depression 33 of channel 32, at which point bearing pad 26 is secured to base 24 and the user feels a positive click indicating that bearing pad 26 is properly mounted on base 24.

Once assembled, base assembly 22 is mounted on first end 42 of handle by sliding male engagement feature (T-shaped projecting track) 54 of handle 40 to female engagement feature (T-shaped channel) 25 in base 24. Referring to FIGS. 1-2, 5-7 and 11A-11B, once base assembly 22 is mounted on handle 40, base adjustment member 94 is aligned with threaded passage 99 and is threadedly engaged in passage 99 by gripping and turning knob 96. The position of base assembly along base axis A_{B} is then adjusted by rotating knob 96. As threaded portion 97 of base adjustment member 94 is rotated, threaded portion moves further into or out of engagement with passage 99, thereby moving base assembly 22 along projecting track 54 and base axis A_{B}. The user may use guide markings 93a, 93b to determine the initial position of base assembly 22 relative to handle 40 and along base axis A_{B}. In this example, inserter device 20 is being used to insert femoral component 10, which is a cruciate retaining (“CR”) femoral component. Accordingly, the user sets the initial position of base assembly by aligning guide marking 93b on handle 40 with guide marking 93a labeled “CR” on base 24.

Turning now to FIGS. 1-2 and 11B, first and second distances D₁, D₂ are set to accommodate femoral component 10 (FIGS. 12 and 13) by manipulating height adjustment member 82. More specifically, the user turns knob 84 in one direction to decrease first and second distances D₁, D₂ and in the other direction to increase first and second distances D₁, D₂. As knob 84 is rotated, threaded portion 87 of adjustment member 82 rotates therewith, threading further into or out of sleeve 88. As threaded portion 87 threads further into or out of sleeve 88, sleeve 88 moves down or up, respectively, within locking lever 76. As a result, sleeve 88 moves pivot bracket 78 downward or upward, respectively, along slot 80 of tracking lever 76. Through its connection to hinge 77, the movement of pivot bracket 78 along slot 80 causes locking rod 60 to move down or up along handle axis A_{H}, thereby adjusting first and second distances D₁, D₂. The user manipulates knob 84 until guide marking 90a in sleeve 88 is aligned with the one of markings 90b labeled “CR” on locking lever 76.

Once base assembly 22 is mounted on handle 40 and initially positioned along base axis A_{B}, and first and second distances D₁, D₂ are initially positioned, the user is now ready to mount femoral component 10 to inserter device 20. Referring to FIGS. 1-2 and 12-13, locking lever 76 is pivoted to the released position shown in FIG. 2. The movement of locking lever 76 to the released position causes locking rod 60 to slide upward along handle axis A_{H}, thereby moving locking rod 60 to the unlocked position wherein locking lip is positioned at second distance D₂ from upper bearing surface 28 of bearing pad 26. As shown in FIGS. 12 and 13, femoral component 10 is positioned atop bearing pad 26 such that articulating surface 14 abuts upper bearing surface 28 and locking end 62 of locking member 60 extends through notch 16. In this position, locking lip 66 of locking member 60 extends over and is substantially parallel to a portion of bone engaging surface 12 adjacent notch 16.

Turning now to FIGS. 1 and 12-13, locking lever 76 is then pivoted to the secured position shown in FIG. 1. As a result, locking lever 76, via connection through hinge joint 77, pulls locking rod 60 downward to the locked position shown in FIGS. 1 and 12-13. In this position, locking lip 66 is positioned at first distance D₁, wherein locking lip 66 bears against bone engaging surface 12 of femoral component 10 proximal notch 16. Further, in this position, femoral component 10 is gripped between locking lip 66 of locking rod 60 and upper bearing surface 28 of bearing pad 26. Inserter device 20 uses material deflection to lock locking lever 76 in the secured position, and thereby secure locking rod 60 in the locked position. More particularly, the offset engagement of hinges 77, 79 relative to locking lever 76, causes deflection portion 81 to deflect or bend slightly, which provides the clamping force that locks locking lever 76 in the secured position of FIGS. 1, 12 and 13. In other words, as illustrated in FIGS. 8, 8A and 9, when locking lever 76 is in the locked position shown in FIG. 8, hinges 77, 79 and 85 are offset from one another; that is, respective pivot points 77a, 79a and 85a are not aligned with one another. As locking lever 76 is moved from the released position, shown in FIG. 9, to the secured position shown in FIG. 8, locking lever 76 reaches a position, shown in FIG. 8A, wherein pivot points 77a, 79a and 85a are aligned with one another to create a resistance break-over point. At this point, deflection of some part of the device is required to allow locking lever 76 to be moved further toward the secured position of FIG. 8. Accordingly, as locking lever 76 is forced toward the secured position, deflection portion 81 deflects or bends slightly to allow locking lever 76 to overcome the resistance break-over point and snap into the secured position of FIG. 8. Although, deflection portion 81 is provided for the deflection purpose, it should be understood that other components of device 20 may be provided with deflection capacity alternative, or in addition to deflection portion 81.

It should be noted that guide markings 90a-b and 93a-b are intended to provide initial settings. If it is too difficult to move locking lever 76 from the released position to the secured position, the user may finely adjust distances D₁, D₂ and/or the position of base assembly 22 on base axis A_{B}, by manipulating height adjustment member 82 and base adjustment member 94, respectively. Such fine adjustments can be made to accommodate various sizes and types of femoral components.

Once femoral component 10 is secured to inserter device 20, device 20 is used to insert femoral component 10 into the incision and position femoral component 10 in the distal end of the femur (not shown). Mounting posts 11 of femoral component 10 are aligned with pre-drilled holes in the femur (not shown) and device 20 is used to force posts 11 into the pre-drilled holes and mount bone engaging surface 14 against the distal end of the femur. A force delivering object, such as a mallet or hammer, may be used to aid in mounting femoral component 10 on the bone. In this case, force is applied to impact receiving surface 48 by the hammer or mallet. The force is transferred from surface 48 down handle 40, through base assembly 22 and to femoral component 10.

Once femoral component 10 is mounted to the femur, locking lever 76 is moved from the secured position of FIGS. 1, 12 and 13 to the released position of FIG. 2 by depressing trigger 92a of spring release lever 92. Spring release lever 92 pivots at handle 40 causing tab 92b to lift outward from handle 40 which, in turn, forces locking lever 76 outward from handle 40. The movement of locking lever 76 to the released position, in turn, releases locking lip 66 from femoral component 10 and device 20 may be removed from the surgical site.

Inserter device 20 may also be used to remove an implanted femoral component. In this case, first and second distances D₁, D₂ and the position of base assembly 22 may be set as described above. With locking lever 76 in the released position shown in FIG. 2, locking lip 66 is inserted between bone engaging surface 12 of femoral component 10 and the bone at a point proximal notch 16. Chamfered edge 68 of locking lip 66 facilitates insertion of lip 66 between femoral component 10 and the bone. Locking lever 76 is then pivoted to the secured position shown in FIG. 1 which, as described above, causes femoral component to be gripped between locking lip 66 and bearing surface 28.

Once inserter device 20 is secured to femoral component 20, an extraction device (not shown), such as a slap-hammer, may be coupled to second end 44 of handle 40 by mating a complementary engagement feature on the extraction device with engagement feature 50 of handle 40. The slap-hammer is used in a conventional manner to apply an extraction force through device 20 and to femoral component 20.

As noted above, when securing femoral component 10 to device 20, device 20 and its locking lip 66 grips femoral component 10 in the area of notch 16 rather than gripping the femoral component 10 at its outermost edges. Thus, device 20 allows the user to insert the femoral component into the surgical site through a relatively small incision and manipulate the femoral component within a small surgical space. Accordingly, inserter device 20 minimizes the size of the incision needed to access the femur and implant the femoral component 10 and minimizes the disruption to and invasion of the surrounding tissue.

The inserter device 20 is described above with reference to its use in inserting a cruciate retaining femoral component. However, as guide markings 90a-b and 93a-b suggest, inserter device 20 may be adjusted using height adjustment member 82 and base adjustment member 94 for use in inserting a posterior stabilized femoral component.

Although the exemplary embodiment illustrated herein and described above is adapted to implant or remove a femoral component, device 20 may be adapted for use in implanting or removing other prostheses, such as tibial trays, prosthetic shoulder components, prosthetic hip components, and other prostheses. In some cases, this may be achieved simply by providing a bearing pad having a different size and shape, but still mountable to base 24. In other cases, base assembly 22, as a whole, may have a different shape.

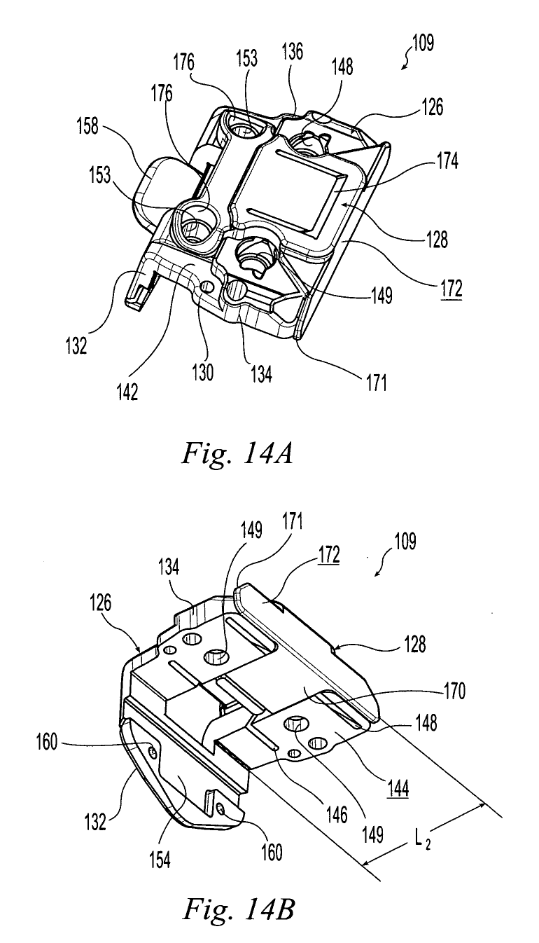

Referring now to FIGS. 14A and 14B, cut guide assembly 109 according to one embodiment of the present invention is illustrated. As discussed in further detail below, cut guide assembly 109 is configured to be placed on resected (i.e. distal cut has been made) distal end F_{D} of femur F and guide in finishing and shaping femur F to receive a femoral implant such as prior art femoral implants 110, 210 and 310 (FIGS. 19A-19C).

Referring to FIGS. 19A and 19C, femoral implants 110 and 310 are cruciate retaining femoral prostheses having similar features including bone engaging surface 112, opposing articulating surface 113, medial side 117 and lateral side 119 opposite medial side 117. Femoral component 110 defines width W extending between medial and lateral sides 117, 119. Bone engaging surface 112 includes posterior surface 114, anterior surface 116 facing posterior surface 114, distal surface 118 extending between medial and lateral sides 117, 119, anterior chamfer surface 120 extending between distal surface 118 and anterior surface 116, posterior chamfer surface 122 extending between distal surface 118 and posterior surface 114, and trochlear surface 124 protruding from anterior chamfer surface 122 and extending between distal surface 118 and anterior surface 116. Articulating surface 113 of femoral implants 110 and 310 forms a pair of posterior condyles 121. Notch 123 extends through implants 110, 310 from bone engaging surface 112 to articulating surface 113 and spaces apart condyles 121. Femoral implant 110 (FIG. 19A) differs from femoral implant 310 (FIG. 19C) in that articulating surface 113 of implant 310 has been extended such that width W₂ of posterior condyles 121 of implant 310 is greater than width W₁ of posterior condyles 121 of implant 110. Thus, implant 310 provides greater range of flexion and is hereinafter referred to as “flex femoral implant” 310. Flex femoral implant 310 is described in further detail in U.S. Pat. No. 6,123,729 to Insall et al., entitled Four Compartment Knee, filed Mar. 10, 1998, assigned to the assignee of the present application and hereby incorporated by reference.

Turning now to FIG. 19B, femoral implant 210 is a posterior stabilized femoral component. Similar to femoral implants 110 and 310, femoral implant 210 includes bone engaging surface 212, opposing articulating surface 214, medial side 217 and lateral side 219 opposite medial side 217. Articulating surface 214 forms a pair of condyles 221. Notch 223 extends through implant 210 from bone engaging surface 212 to articulating surface 213 and femoral implant 210 spaces apart condyles 221. Similar to femoral implants 110 and 310, bone engaging surface 212 of femoral implant 210 includes posterior surface 24, anterior surface 216, distal surface 218, anterior chamfer surface 220, posterior chamfer surface 222 and trochlear surface 224. Femoral implant 210 differs from implant 110 in that it includes femoral box 225 protruding outwardly from distal surface 218 and extending along the edge of notch 223.

Referring now to FIGS. 19A-19C, anterior chamfer surfaces 120, 220 extend from distal surfaces 118, 218, respectively, at angle α_{A} (FIG. 19A), while posterior chamfer surfaces 122, 222 extend from distal surfaces 118, 218, respectively, at angle αₚ (FIG. 19A). Referring to FIG. 19A, standard cruciate retaining femoral implant 110 defines length L_{ST} extending between anterior and posterior surfaces 114, 112. Referring to FIG. 19C, flex cruciate retaining femoral implant 310 defines length L_{FL} extending between anterior and posterior surfaces 114, 112. Length L_{ST} is greater than L_{FL} due to the increased width of W₂ relative to W₁.

Turning back to FIGS. 14A and 14B, the features of cut guide assembly 109 will now be discussed. Cut guide assembly 109 generally includes chamfer guide 126 and trochlear guide 128, a portion of which is removably nested, or interconnected, with chamfer guide 126.

Referring to FIGS. 14A-B and 15-16, chamfer guide 126 includes distal portion 130 and anterior portion 132. Distal portion 130 includes opposing medial and lateral ends 134, 136; opposing anterior and posterior edges 138, 140 extending between medial and lateral ends 134, 136; and opposing distal and proximal faces 142, 144 extending between both anterior and posterior edges 138, 140 and medial and lateral ends 134, 136. Proximal face 144 is configured for placement against the resected distal end F_{D} of femur F, as discussed in further detail below. Distal portion 130 includes anterior chamfer slot 146 and posterior chamfer slot 148 extending therethrough from proximal face 144 to distal face 142. Anterior chamfer slot 146 extends through distal portion 130 relative to distal face 142 at an angle corresponding to angle α_{A}. Posterior chamfer slot 148 extends through distal portion 130 relative to distal face at an angle corresponding to angle αₚ. Anterior and posterior chamfer slots 144, 146 are configured to receive and guide a saw in making the anterior and posterior chamfer cuts of femur F. Although anterior and posterior chamfer slots 144, 146 are illustrated in the form of elongated slots, distal portion 130 could alternatively be provided with one or more enlarged openings that form angled anterior and posterior chamfer guide surfaces for guiding a saw.

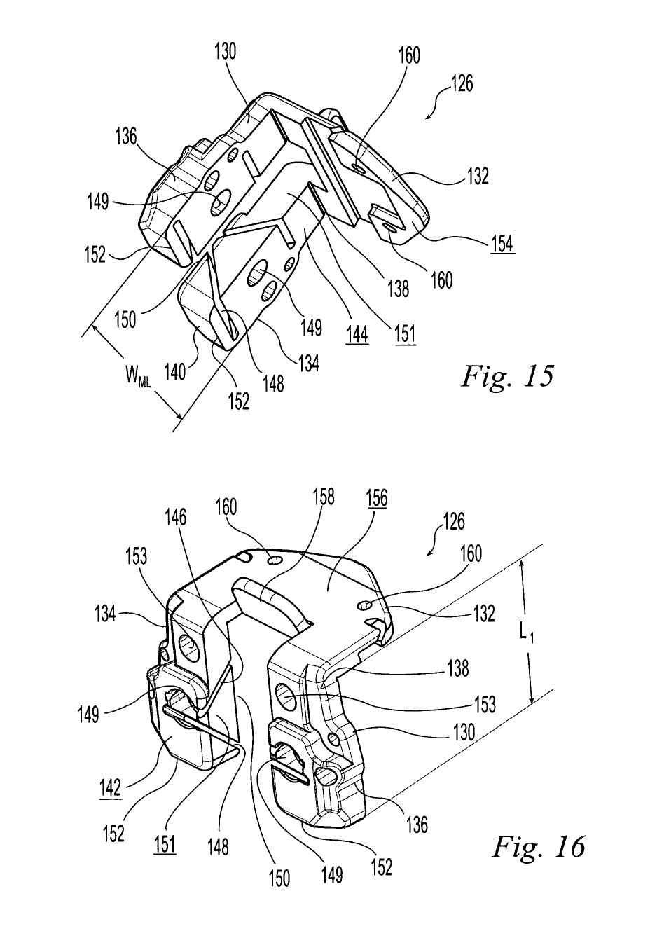

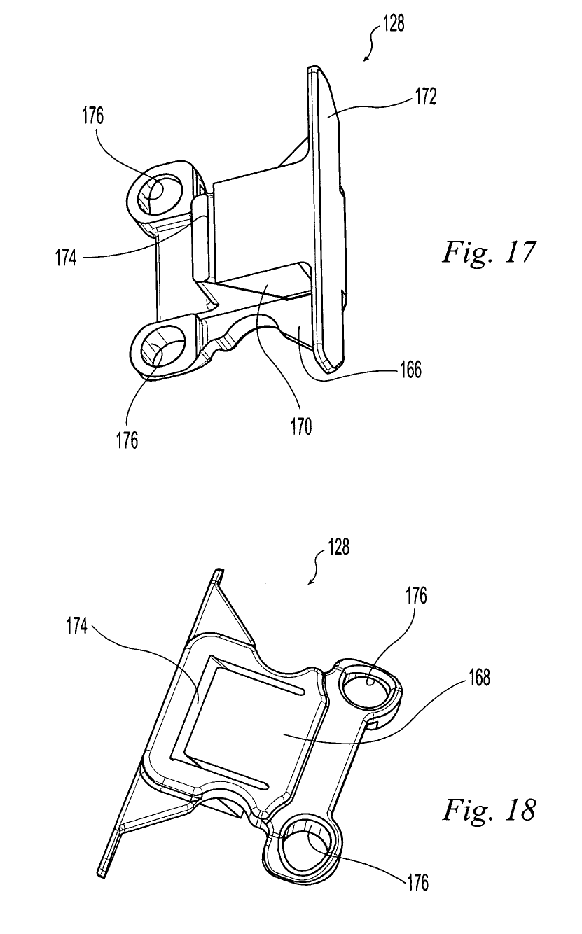

Referring still to FIGS. 15-16, distal portion 130 includes a pair of spaced apart arms 152 defining box cut guide opening 150 therebetween. Box cut guide opening 150 extends through distal portion 130 from proximal face 144 to distal face 142. Box cut guide opening 150 is configured to correspond to the geometry of femoral box 225 of femoral implant 210 (FIG. 19B). Distal portion 130 defines a medial-lateral width W_{ML} extending between medial and lateral ends 134, 136. Width W_{ML} may correspond to width W of implant 110 (FIG. 19A) to provide a reference for the user to observe where the medial and lateral sides 117, 119 of the implant will be positioned. Distal portion 130 also includes drill guide bores 149 extending therethrough from proximal face to distal face. Guide bores 149 are configured to guide a drill in making holes in distal end F_{d} of femur F to receive mounting posts 115, 215 of implants 110, 210, 310. Distal portion 130 includes a pair of threaded handle receiving openings 153 extending into distal face 142. Openings 153 are adapted to threadedly receive a handle (not shown) and are spaced apart from one another to allow the user to manipulate the cut guide assembly 109 in either a medial or lateral approach, as discussed further below.

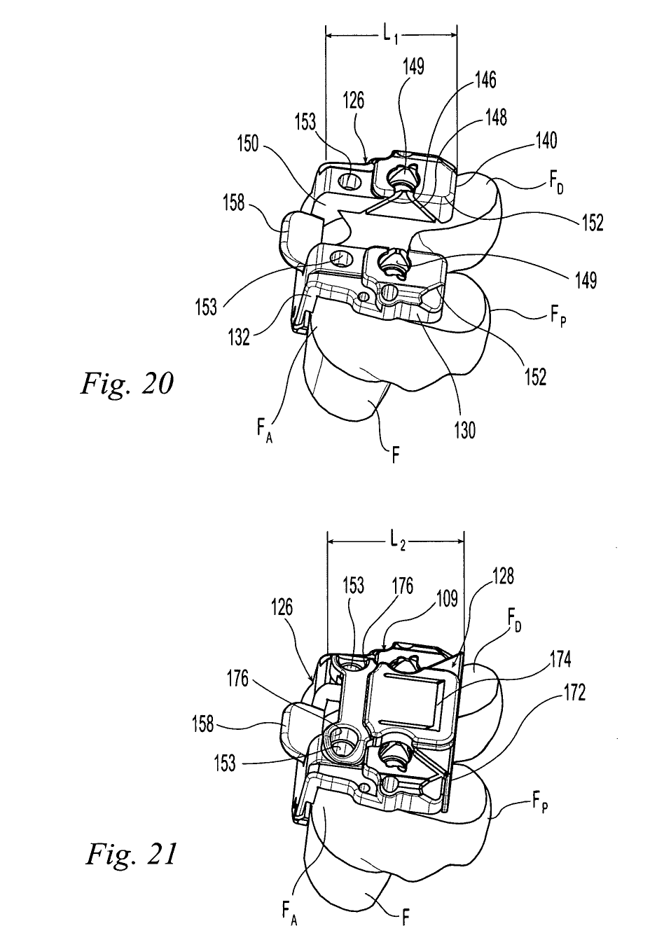

Referring to FIGS. 14A-B and 15-16, anterior portion 132 extends from proximal face 144 of distal portion 130, and includes inside surface 154 and outside surface 156. Inside surface 154 is configured to bear against anterior side F_{A} of femur F (FIG. 20). Guide ledge 158 protrudes from outside surface 156 and is aligned with box-cut guide opening 150 of distal portion 130. Anterior portion 132 includes fastener receiving openings 160 extending therethrough and is adapted to receive fasteners (not shown) to secure chamfer guide assembly 109 to femur F (FIG. 20). Distal portion 130 defines a first anterior-posterior length L₁ extending the length of proximal face 144 between between inside surface 154 of anterior portion 132 and posterior edge 140 of distal portion 130. Length L₁ corresponds to length L_{FL} of flex femoral implant 210 (FIG. 19C).

Turning now to FIGS. 14A-B and 17-18, trochlear guide 128 includes lower surface 116 and opposite upper surface 168. Projection 170 extends outwardly from lower surface 166 and is sized and configured to nest within box-cut guide opening 150 of chamfer cut guide 126 (FIGS. 14A and 16). Trochlear guide 128 includes posterior flange 171 that forms posterior edge 172. Posterior flange 171 is configured to overlie posterior edge 140 of chamfer guide 126 when trochlear guide 128 is interconnected with chamfer guide 126 (FIGS. 14A and 15). Cut guide assembly 109 defines a second anterior-posterior length L₂ extending inside surface 154 of anterior portion 132 and posterior edge 172 of trochlear guide 128. Second anterior-posterior length L₂ corresponds to length L_{ST} of standard cruciate retaining implant 110.

Trochlear cut guide 128 also includes trochlear cut guide surface 174 in the form of a captured, U-shaped slot extending through trochlear guide 128 from upper surface 118 to lower surface of projection 170. Trochlear cut guide surface 174 is configured to receive and guide a saw in making the trochlear cuts. Trochlear cut guide surface 174 need not be in the form of a captured slot, but may take any form suitable for providing guide surfaces for a saw or other cutting or milling instrument to make a cut in femur F to accommodate trochlear surface 124, 224 of implants 110, 210, 310 (FIGS. 19A-C). Trochlear cut guide 128 also includes handle receiving hole 176 extending therethrough from upper surface 168 to lower surface 166. Handle receiving hole 176 is configured to align with handle receiving opening 153 when trochlear guide 128 is interconnected to chamfer guide 126.

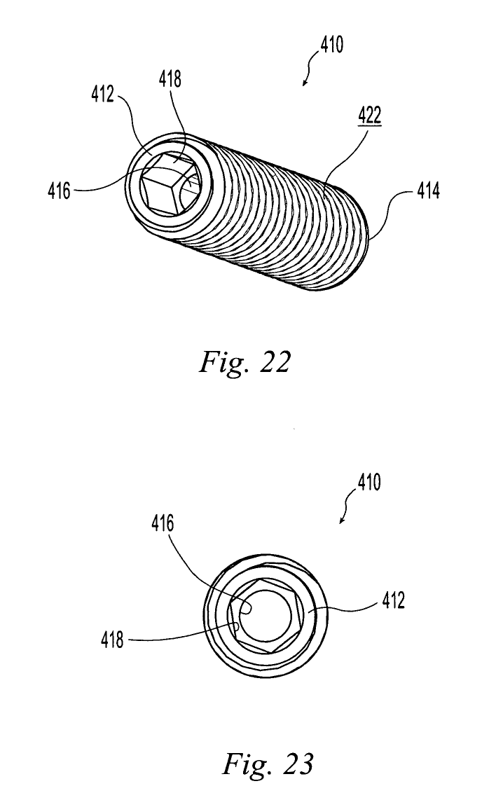

Referring now to FIGS. 14A-14B and 20-21, the operation of cut guide assembly will now be described. After determining which femoral implant 110, 210, 310 (FIGS. 19A-19C) should be implanted, the surgeon prepares distal end F_{D} of femur F by making a distal cut of distal end F_{D} using any conventional means. Next, the surgeon assembles cut guide assembly 109 by inserting projection 170 of trochlear guide 128 into box-cut guide opening 150 of chamfer guide 126 such that handle receiving hole 176 is aligned with threaded handle receiving opening 153 and flange 171 overlies posterior edge 140. A threaded handle (not shown) is then inserted through one of handle receiving holes 176 of trochlear guide 128 and threadedly engaged in the aligned handle receiving opening 153 of chamfer guide 126 to secure trochlear guide 128 to chamfer guide 126. As noted above, holes 176 and openings 153 are positioned medially and laterally to allow the surgeon to select either set to accommodate either a medial or lateral approach.

Once guide assembly 109 is assembled, the handle (not shown) is used to position cut guide assembly 109 on distal end F_{D} of femur F such that proximal face 144 and inside surface 154 of chamfer guide 126 respectively bear against distal end F_{D} and anterior side F_{A} of femur F. At this point, because medial-lateral width W_{ML} of cut guide assembly 109 corresponds to width W of implant 110 (implants 210 and 310 may also have the same width as width W), the surgeon may observe the prospective positioning of the medial and lateral sides 117, 119 of implant 110 and adjust as needed. Once positioned on femur F, cut guide assembly 109 may be secured to femur F by inserting pins (not shown) through fastener receiving openings 160 in chamfer guide 126.

Once cut guide assembly 109 is secured to femur F, cut guide assembly 109 may be used to guide the finishing cuts, namely the posterior cut, trochlear cut, anterior chamfer cut, posterior chamfer cut and box cut as needed and in any order desired. For instance, a saw may be inserted along trochlear cut guide surface 174 and into the bone to cut the femur F and provide a surface and geometry complementary to trochlear surface 124, 224 of implants 110, 210, 310. If the surgeon is implanting standard cruciate retaining femoral implant 110 or standard posterior stabilized femoral implant 210, posterior edge 172 of trochlear guide 128 is used as a guide surface to guide the saw in cutting posterior side Fₚ of femur F. Because second anterior-posterior length L₂ corresponds to length L_{ST} (the anterior-posterior length of standard posterior cruciate implant 210 may also correspond to length L_{ST}) the distal end F_{D} of femur F would now have the proper anterior-posterior length to receive implant components 110, 210. On the other hand, if the surgeon selects flex femoral implant 310, the posterior cut of femur F is either deferred or a preliminary posterior cut is made using posterior edge 172 as a guide.

Next, handle (not shown) is disengaged from threaded opening 153 and removed from hole 176. Trochlear guide 128 is removed from its nested, interlocked position with chamfer guide 126 leaving only chamfer guide 126 mounted on distal end F_{D} of femur F as illustrated in FIG. 20. If flex femoral implant 310 is being implanted, the posterior cut (or final posterior cut if preliminary cut was made) of femur F is now made using posterior edge 140 of chamfer guide 126 to guide the saw. Because first anterior-posterior length L₁ corresponds to length L_{FL} of flex femoral implant 310, distal end F_{D} of femur F would now have the proper anterior-posterior length to receive implant 310.

Next, anterior and posterior chamfer guide slots 146, 148 are used to guide a saw in making chamfer cuts of femur F to provide anterior and posterior chamfer surfaces in femur F that correspond to anterior and posterior chamfer surfaces 120/220, 122/220, respectively, of implants 110, 210, 310 (FIGS. 19A-19C).

If the surgeon has selected posterior cruciate femoral implant 210 (FIG. 19B), the surgeon now makes the posterior box cuts using surfaces 151 of box cut opening 150 and guide ledge 158 to guide the saw or milling instrument. The resulting femoral box (not shown) in femur F has a geometry complementary to that of femoral box 225 and may receive femoral box 225 of implant 210.

Once all the necessary cuts are made, handle (not shown) may be re-engaged to threaded hole 153. The pins (not shown) used to secure assembly 109 to the bone are removed and chamfer guide 126 is removed from femur F using handle (not shown) the resulting distal end F_{D} of femur F is now shaped to receive one of implants 110, 210, 310.

Turning now to FIGS. 22-25, positioning device or sleeve 410 according to one embodiment of the present invention will now be described. Positioning device 410 is adapted for use in positioning an implant or instrument against a bone. Referring particularly to FIGS. 22 and 23, positioning device 410 is elongate and includes first end 412 and opposite second end 414. Passage 416 extends through device 410 from first end 412 to second end 414. Passage 416 is sized and configured to receive a fastener (not shown), such as a pin, nail, screw, or peg, for attaching to bone. Positioning device 410 includes recess 418, which extends into first end 412 and is coaxial with, and in communication with, passage 416. Recess 418 is adapted to receive a drive tool (not shown), such as a drill, screwdriver, wrench or ratchet. More particularly, recess 418 has a hexagonal cross-sectional shape. Although recess 418 is illustrated as having a hexagonal shape, recess 418 may have any shape capable of mating with a drive tool. Positioning device 410 also has threaded external surface 422. Positioning device 410 is sized in length and cross-section to be received in a threaded opening in the implant or instrument, which is being positioned against the bone.

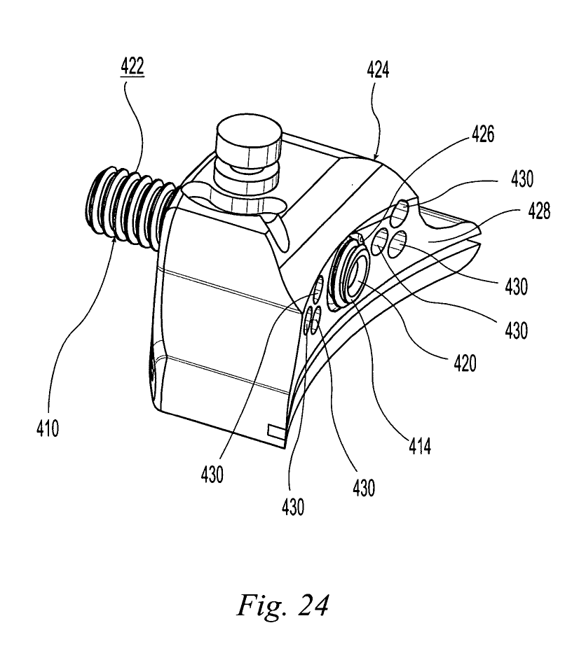

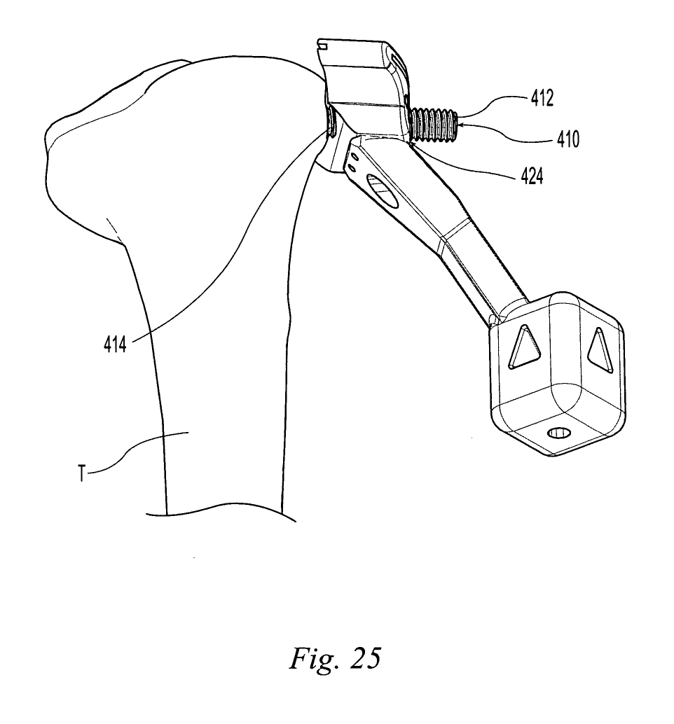

FIGS. 24 and 25 illustrate a use of positioning device 410 in positioning tibial cut guide instrument 424 against tibia bone T. The use of tibial cut guide 424 is more fully described in U.S. Patent Publication No. 2004/0153066, filed as U.S. application Ser. No. 10/357,282 to Coon et al., entitled Apparatus for Knee Surgery and Method of Use, assigned to the assignee of the present application and hereby incorporated by reference. Tibial cut guide 424 is positioned in the desired position adjacent and/or against the surface of tibia T using any means including those described in the above-mentioned publication. Positioning device 410 is threadedly engaged with threaded receiving opening 426 in tibial cut guide 424. Positioning device 412 is driven further into opening 426 using a drive tool until positioning device 412 protrudes from opening 426 proximal bone engaging surface 428, as shown in FIG. 24, and abuts tibia T, as shown in FIG. 25. Next, a fastener, such as a pin, is inserted through passage 416 and into tibia T. Additional fasteners may be driven through receiving holes 430 and into tibia T to further secure cut guide 424 to tibia T. Furthermore, additional threaded holes may be provided in cut guide 424 to accommodate additional positioning devices 410.

Positioning device 410 acts as a bushing occupying space between the surface of tibia T and bone engaging surface 428 of guide 424. Positioning device 410 serves to improve stability and positioning of guide 424 against irregular bony surfaces where portions of surface 428 of guide 424 are spaced from the surface of tibia T when guide 424 is placed against tibia T.

It should be noted that positioning device 410 may be used with any instrument or implant that requires stable fixation to a bone. Furthermore, although the illustrated embodiment shows positioning device 410 as having a female hexagonal tool engagement recess 418, device 410 may be adapted such that the external surface at first end 412 forms a male engagement member engageable with a female member on a drive tool.

Also, in one embodiment, the pin or other fastener may have a head larger than the diameter of passage 416 and recess 418 such that the head of pin does not enter passage 416 and remains accessible. Alternatively, the head of the pin may be larger than the diameter of passage 416 but smaller than recess 418, such that the head of the pin may be driven into recess 418 but not into passage 416. In this case, recess 418 may still be engaged by the drive tool when the head of the pin is in recess 418. Thus, when removal of the guide or instrument is desired, the pin may be removed from the bone by driving device 410 out of engagement with the threaded hole.

In addition, positioning device 410 may be captured in threaded opening 426 such that positioning device 410 may be allowed to threadably travel along opening 426 but may not be completely removed form opening 426.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A device for inserting an implant into an end of a bone and/or for removing the implant from the end of the bone, the implant having a proximal surface configured to be positioned against the end of the bone and an opposite distal surface, the implant including a notch extending therethrough from the proximal surface to the distal surface, said device comprising:
a base assembly including an upper bearing surface configured to bear against the distal surface of the implant, said base assembly including an opening extending therethrough, said opening positioned to align with the notch when said upper bearing surface bears against the distal surface of the implant;
a handle having a first end coupled to said base assembly and an opposite second end;
a locking member slidably coupled to said handle and having a locking end extending from said handle and through said opening of said base assembly, said locking end extending through the notch of the implant when said upper bearing surface bears against the distal surface of the implant, said locking end having a locking lip extending transversely from said locking member, said locking member movable between a locked position wherein said lip is positioned at a first distance from said upper bearing surface and an unlocked position wherein said lip is positioned at a second distance from said upper bearing surface, said second distance being greater than said first distance, and wherein when said locking member is in said locked position said lip is engagable with the proximal surface of the implant at a location proximal the notch thereby gripping the implant between said upper bearing surface and said lip; and
a locking actuator operatively engaged with said locking member, said locking actuator operable to move said locking member between said locked position and said unlocked position.

2. The device of claim 1 wherein said handle includes an elongate handle body extending between said first end and said opposite second end and defining a handle axis, and wherein said locking member is slidable relative to said handle along said handle axis.

3. The device of claim 2 wherein said handle body defines a central passage extending along said handle axis, and said locking member comprises an elongate locking rod slideably disposed within said passage.

4. The device of claim 2 wherein said base assembly is slideably coupled to said first end of said handle, said base assembly slideable relative to both said handle and said locking member along a base axis, wherein said base axis is transverse to said handle axis.

5. The device of claim 4 further including a base adjustment member operably coupled to said base assembly, said base adjustment member operable to secure said base assembly in position along said base axis.

6. The device of claim 2 wherein said second end of said handle includes an impact receiving surface.

7. The device of claim 2 further including an extractor instrument removably coupled to said second end of said handle.

8. The device of claim 1 wherein said base assembly includes a base slideably coupled to said first end of said handle and a bearing pad removably coupled to said base, said bearing pad including said upper bearing surface, said upper bearing surface being contoured to mate with the distal surface of the implant.

9. The device of claim 1 wherein said locking lip includes a chamfered edge.

10. The device of claim 1 wherein said locking actuator includes a height adjustment member operably engaged with said locking member, said height adjustment member operable to adjust said first and second distances.

11. The device of claim 1 wherein said locking actuator includes a locking lever pivotally coupled to said handle and operably engaged with said locking member, said locking lever pivotable between a secured position wherein said locking member is secured in the locked position and a released position wherein said locking member is released from said locked position.

12. The device of claim 11 further comprising an actuator release member coupled to said handle and operable to release said locking lever from said secured position.

13. The device of claim 1 wherein said handle defines a handle axis extending between said first end and an opposite second end, said locking member being slidable relative to said handle along said handle axis, and wherein said base assembly is slideably coupled to said first end of said handle, said base assembly slideable relative to both said handle and said locking member along a base axis, said base axis being transverse to said handle axis.

14. A device for inserting an implant into the end of a bone and/or for removing the implant from the prepared end of the bone, the implant having a proximal surface for placement against the bone and an opposite distal surface comprising:
an elongate handle extending between a first end and a second end and defining a handle axis;
a base assembly slideably coupled to said first end, said base slideable relative to said handle along a base axis, said base axis being transverse to said handle axis, said base including an upper bearing surface configured to bear against the distal surface of the implant;
an elongate locking member extending between a locking end and an opposite free end, said locking member slidebly coupled to said handle and slideable relative to said handle along said handle axis, said locking member having a locking end extending outwardly from both said handle and said base assembly, said locking end having a locking lip extending transversely from said locking member, said locking member movable between a locked position wherein said locking end extends outwardly from said upper bearing surface at a first distance and an unlocked position wherein said locking end extends outwardly from said upper bearing surface at a second distance, said second distance being greater than said first distance, and wherein when said locking member is in said locked position said lip is engagable with the proximal surface of the implant, whereby the implant is gripped between said upper bearing surface and said lip; and
a locking actuator operatively engaged with said locking member, said locking actuator operable to move said locking member between said locked position and said unlocked position.

15. The device of claim 14 wherein said base assembly includes a base slideably coupled to said first end of said handle and a bearing pad removably coupled to said base, said bearing pad including said upper bearing surface.

16. The device of claim 15 wherein said upper bearing surface is contoured to mate with the distal surface of the implant.

17. The device of claim 16 wherein said bearing pad is formed of plastic.

18. The device of claim 14 wherein said locking actuator includes a height adjustment member operably engaged with said locking member, said height adjustment member operable to adjust said first and second distances.

19. The device of claim 14 wherein said locking actuator includes a locking lever pivotally coupled to said handle and operably engaged with said locking member, said locking lever pivotable between a secured position wherein said locking member is secured in the locked position and a released position wherein said locking member is released from said locked position.

20. A device for inserting a femoral component into the end of the bone and/or for removing the femoral component from the end of the bone, the femoral component having a proximal surface configured to be positioned against the end of the bone and an opposite articulating distal surface, the femoral component including an intracondylar notch extending therethrough from the proximal surface to the distal surface, said device comprising:
a base assembly including an upper bearing surface configured to bear against the articulating distal surface of the femoral component, said base assembly including an opening extending therethrough, said opening aligned with the notch when said upper bearing surface bears against the articulating distal surface;
an elongate handle defining a handle axis and having a first end coupled to said base assembly and an opposite second end; and
a locking member slidebly coupled to said handle and slideable relative to said handle along said handle axis, said locking member having a locking end extending from said first end of said handle and through said opening of said base assembly, said locking end extending through the notch when said upper bearing surface bears against the articulating distal surface, said locking member having a locking lip extending transversely from said locking end, said locking member movable between a locked position wherein said lip is positioned at a first distance from said upper bearing surface and an unlocked position wherein said lip is positioned at a second distance from said upper bearing surface, said second distance being greater than said first distance, and wherein when said locking member is in said locked position said lip engages the proximal surface at a location proximal the notch and grips the femoral implant between said upper bearing surface and said lip.

21. The device of claim 20 further including a locking actuator operatively engaged with said locking member, said locking actuator operable to move said locking member between said locked position and said unlocked position.
